(19) United States
(12) Patent Application Publication
(10) Pub. No.: US 2008/0318909 A1
(43) Pub. Date: Dec. 25, 2008

(54) Use Of Linoleic Compounds Against Heart Failure

(76) Inventors: Genevieve C. Sparagna, Louisville, CO (US); Adam J. Chicco, Fort Collins, CO (US); Russell L. Moore, Louisville, CO (US); Sylvia A. McCune, Loveland, CO (US)

Correspondence Address: LATHROP & GAGE LC, 4845 PEARL EAST CIRCLE, SUITE 300, BOULDER, CO, 80301, US

(21) Appl. No.: 12/140,088

(22) Filed: Jun. 16, 2008

Related U.S. Application Data
(60) Provisional application No. 60/944,032, filed on Jun. 14, 2007.
(60) Provisional application No. 60/944,045, filed on Jun. 14, 2007.

(51) Int. Cl. A61K 31/201; A61P 9/00; A61K 31/683

(52) U.S. Cl. 514/148; 514/560

(57) ABSTRACT

Linoleic acid and related cardiolipin products are used as dietary supplements that provide cardiac benefits against a variety of cardiac related symptoms and diseases. For example, the disclosed compositions and methods may be used to treat or prevent hypertension, ischemic cardiomyopathy, heart disease, Barth Syndrome and heart failure.

### RELATED APPLICATIONS

The present application claims the benefit of priority to U.S. Provisional Patent Application Ser. Nos. 60/944,032 and 60/944,045, both filed Jun. 14, 2007. Each of these applications is incorporated herein by reference.

### GOVERNMENT RIGHTS

The United States Government has rights in this invention under Contract No. 5R21HL084129-02 between the National Institutes of Health (NIH) and the University of Colorado.

### BACKGROUND

Heart failure is a condition that can result from any structural or functional cardiac disorder that impairs the ability of the heart to fill with blood or pump a sufficient amount of blood through the body. Heart failure differs from heart attack (myocardial infarction) which is the cessation of normal cardiac function, or cessation of heartbeat with subsequent hemodynamic collapse leading to death. It is known that during heart failure (HF), mitochondria cannot produce an adequate supply of ATP to support the demands on the heart [1, 2]. However, the role of lipids in this process is poorly understood.

Cardiolipin (CL), as shown in FIG. 1, is a unique phospholipid in the mitochondria that is required for the proper function of numerous mitochondrial enzymes involved in oxidative phosphorylation. It is optimally functional in the heart only when it contains four linoleic acid (linoleate) side chains in a form known as (18:2)₄CL. The amount of (18:2)₄CL decreases, and alternate molecular species of CL increase, during the progression to HF in both rats and humans (FIGS. 4, 5) [3]. Furthermore, the decrease in (18:2)₄CL correlates with a decreased activity of the electron transport enzyme cytochrome c oxidase (complex IV).

CL is a major cardiac phospholipid found almost exclusively in the mitochondrial inner membrane where it is essential for the optimal function of several key enzymes involved in mitochondrial energy metabolism [5]. A CL-rich inner membrane environment is required for the proper assembly, structure, and function of the mitochondrial respiratory chain complexes involved in the oxidative generation of ATP [6, 7]. In addition to its support of the enzymes involved in electron transport, CL has been proposed to participate directly in proton conduction through cytochrome bc₁ [8], and the electrostatic anchoring of cytochrome c to the inner mitochondrial membrane [9]. Hence, CL has an involvement in the cytochrome c release which triggers downstream events in apoptosis [4, 10].

Unlike most membrane phospholipids that have a single glycero-phosphate backbone and two fatty acyl side-chains, CL has a double glycerophosphate backbone and four fatty acyl (FA) side-chains. In eukaryotes, CL is biosynthesized from phosphatidylglycerol (PG) and cytidinediphosphate-diacylglycerol (CDP-DAG) by CL synthase in the inner mitochondrial membrane, as shown in FIG. 2 [5]. In a healthy mammalian heart, the CL acyl side-chain distribution is approximately 80-90% linoleic acid (18:2), <10% oleic acid (18:1), and <10% linolenic acid (18:3), with trace amounts of palmitoleic acid (16:1) and arachidonic acid (20:4). The designations (18:2), (18:1), (18:3), (16:1), (20:4), etc., refer to the number of carbon atoms in the fatty acid chain and the number of double bonds therein. As used herein, the designations sometimes take the place of the corresponding fatty acid common name. For example, linoleic acid (18:2) contains 18 carbon atoms and 2 double bonds, and is sometimes referred to as simply “18:2”.

The 18-carbon unsaturated acyl side-chain composition is an essential structural feature required for the high affinity binding of CL to membrane proteins [11], and an 18:2-rich configuration, in particular seems important for maintenance of mitochondrial respiration [12]. The enzymes involved in de novo PG and CL synthesis do not exhibit acyl-specificity [13, 14], therefore the 18:2-rich composition of CL is achieved via an acyl chain remodeling process that is not completely understood [5, 15]. Recent studies indicate that CL is enriched with 18:2 acyl chains by at least two enzyme-dependent pathways (FIG. 3). One pathway, which is shown in FIG. 3, involves a two-step deacylation-reacylation process whereby nonspecific acyl chains were cleaved from CL by a yet undefined phospholipase A₂ (PLA₂) isoform, generating monolysocardiolipin (MLCL; (acyl)₃-CL), followed by the reacylation of MLCL with 18:2 by a mitochondrial MLCL acyltransferase (MLCLAT) preferentially utilizing 18:2-CoA as a substrate [5]. A second remodeling pathway (left side of FIG. 3) involves the transfer of 18:2 acyl chains from phosphatidylcholine (PC) or phosphatidylethanolamine (PE) to (acyl)₄-CL by a mitochondrial linoleoyl-specific transacylase, generating (18:2)1-(acyl)₃-CL [16]. These processes presumably continue until CL is enriched with 18:2, generating the optimal tetra-linoleoyl species ((18:2)₄CL), provided sufficient 18:2 sources are available.

Rats fed diets deficient in 18:2, an essential fatty acid, had markedly depressed levels of (18:2)₄CL and reduced mitochondrial respiratory activity [12], suggesting that the composition and functional integrity of CL is sensitive to the fatty acid content of the diet. In particular, heart muscle contains more mitochondria than other tissue types and CL alterations may be associated with myocardial pathologies and heart failure. Recently, it was discovered that the primary mechanism responsible for the x-linked cardioskeletal myopathy known as Barth Syndrome (characterized by infantile or childhood onset of dilated cardiomyopathy) is a specific deficiency of tafazzin (TAZ), a mitochondrial CL transacylase, as shown in the remodeling pathway on the left side of FIG. 3, which is believed to result in aberrant CL remodeling and mitochondrial dysfunction [17, 18]. When fibroblasts from Barth Syndrome patients were supplemented with 18:2, the amount of (18:2)₄CL increased. This indicates that, even with a deficient remodeling gene, these cells were able to make (18:2)₄CL (likely from (18:2)₂PG) and increase their total amount of (18:2)₄CL [19]. Alterations in the myocardial content and/or composition of CL have also been implicated in the mitochondrial dysfunction associated with several other cardiac pathologies including heart failure [3], ischemia reperfusion (IR) injury [20], diabetes and the aging-induced decline in cardiac function.

Early evidence of CL alterations in the failing heart was provided by O'Rourke and Reibel (1992) who reported a reduction in the 18:2 content of CL fractions isolated from hearts induced to failure by chronic aortic banding [21]. More recently, Nasa et al. reported a decrease in 18:2 and increase in 20:4 in phospholipid fractions obtained from viable myocardial tissue of failing hearts twelve weeks after coronary artery ligation [22]. These authors proposed that alterations in the acyl side chain composition of myocardial phospholipids may contribute to the cardiac dysfunction and/or myocardial remodeling in heart failure. To further illustrate the importance of CL composition, a recent study was performed with ischemic cardiomyopathy HF patients with implanted left ventricular assist devices (LVADs) that greatly improved heart function. When CL was studied using fluorescence spectrosopy, it was found that there was no change with or without the LVAD in the total amount of (18:2)₄CL but there was an increase back to control levels of the ratio of (18:2)₄CL to other alternate CL species. This “reverse remodeling” was sufficient to restore functionality to these hearts even though total CL levels were still decreased [23]. The pathophysiological significance of these alterations in CL remains to be fully established.

In both humans and rats, there are only two essential fatty acids: linoleic acid (18:2), an ω-6 fatty acid, and α-linolenic acid (18:3), an ω-3 fatty acid. These fatty acids cannot be made by the body and must be ingested through the diet. Since α-linolenic acid is an omega-3 fatty acid, a lot of attention has been given to dietary guidelines for its inclusion in a healthy diet, and its promotion of anti-inflammatory, anti-thrombotic and anti-arrhythmic properties and improved insulin sensitivity [24, 25]. Interestingly, 18:2 is an ω-6 fatty acid; ω-6 as a class of polyunsaturated fatty acids are routinely viewed in a negative light since they may serve as a precursor of arachidonic acid (20:4), and are routinely associated with promoting inflammation, thrombosis and insulin resistance [25-27]. However, recent evidence has shown that 18:2 actually decreases thrombosis [24], decreases arrhythmias [28] and improves insulin sensitivity [29], and has been shown to prevent mortality from cardiovascular disease in humans [30].

A major source of 18:2 which does not contain a significant quantity of saturated fatty acids, high linoleic safflower oil, is rarely produced today. Since the late 1970s, high linoleic safflower oil has been replaced by the more shelf-stable high oleic safflower oil, which is commonly used in high-end processed foods.

In addition to being high in linoleic acid, safflower oil (both high linoleic and high oleic) has one of the highest levels of α-tocopherol of any natural oil. Vitamin E describes a group of eight naturally occurring compounds: four tocopherols and four tocotrienols. Of these, α-tocopherol is the most bioactive. In fact, α-tocopherol has been described as the most potent lipid-soluble antioxidant in vivo, because it prevents the propagation of lipid oxidation in polyunsaturated fatty acids and lipoproteins and therefore acts to protect lipids by preventing lipid oxidation. α-Tocopherol is associated with prevention of cardiovascular disease as well as a host of other diseases. α-Tocopherol has a role in cellular signaling independent of its antioxidant role by inhibiting protein kinase C, leading to suppressive effects on cytokines and many other inflammatory molecules.

Laaksonen et al. found that intake of monounsaturated fatty acids such as oleate (18:1) did not decrease the risk of mortality from cardiovascular disease [30]. Since linoleic acid (18:2) is an essential fatty acid in rats and humans (Table 1), it may be ingested in the diet in order for (18:2)₄CL to be made and mitochondria to function optimally.

TABLE 1
Characteristics of the major fatty acids

| Carbons:Double | | | |
|---|---|---|---|
| Bonds | Name | Saturation | Comments |
| 16:0 | palmitic acid | saturated | |
| 18:1 | oleic acid | monounsaturated | |
| 18:2 | linoleic acid | ω-6 polyunsaturated | Essential |
| 18:3 | α-linolenic acid | ω-3 polyunsaturated | Essential |
| 20:4 | arachidoinic acid | ω-6 polyunsaturated | |

Linoleic acid is required for the synthesis of arachidonic acid. The conversion is via Δ6 and Δ5 desaturase [31]. These enzymes are also involved in elongation of α-linolenic acid to EPA and DHA. Arachidonic acid is the precursor for many inflammatory and vasodialatory molecules, so its decrease may be beneficial. Furthermore, when arachidonic acid rises in HF, it comprises a greater amount of the fatty acid side chains on cardiolipin causing cardiolipin to be dysfunctional.

Yamaoka et al. found that a diet high in 18:2 (20% corn oil with 18:2>60% of fatty acid content) led to a 86% enrichment of 18:2 in a cardiac CL fraction, whereas a diet low in 18:2 (20% sardine oil, 18:2<7% of fatty acid content), led to 14% 18:2 content in CL fractions after 30 days of feeding [12]. As expected, cytochrome oxidase activity and mitochondrial oxidative phosphorylation was substantially depressed following the low-18:2 diet compared to the 18:2-rich diet.

Peroxisome proliferator-activated receptors (PPARs) are a family of nuclear transcription factors comprised of PPARα, PPARγ, and PPARβ/δ. They have been shown to modulate genes that regulate lipid and glucose metabolism [31]. PPARα, and PPARγ have been studied in the heart where PPARα regulates genes involved in β-oxidation of fatty acids and PPARγ regulates lipid storage. Both of these factors are involved in CL metabolism. PPARα has been shown to activate key enzymes involved in CL synthesis and remodeling: (refer to FIGS. 2 and 3) PGP synthase, CDP-DAG synthase, and PLA₂ [32]. An agonist of PPARγ, the Type-2 diabetes drug Rosiglitazone®, caused large increases in the 18:2 content of CL in the hearts of diabetic mice [33]. Not only are the α and γ isoforms of PPAR involved in CL synthesis/remodeling, but the natural activators for PPARs are polyunsaturated fatty acids, such as 18:2. Furthermore, PPARs are downregulated during pathological hypertrophy leading to HF, which may be a protective mechanism to preserve energy [31]. To date, no studies of PPAR have utilized the SHHF (Spontaneous Hypertension and Heart Failure) animal model.

The SHHF/Mcc-facp (SHHF) rat is a genetic model that has been selectively bred for Spontaneous Hypertension and Heart Failure [34]. The SHHF colony carries the cp gene, an allele of the Zucker fa gene. The cp gene is the result of a nonsense point mutation in the leptin receptor and causes the failure of the production of a functional leptin receptor [35]. Reproducible spontaneous hypertension and congestive heart failure have now been maintained for over twenty one years (forty six generations). All SHHF rats (one hundred percent) develop hypertension and eventually die of HF. The age of onset of hypertension is approximately three to four months, and stable hypertension is seen by five months of age. Homozygote lean SHHF males develop HF at sixteen to twenty-two months of age. Rats in HF have significantly enlarged hearts, congested lungs, edema, peritoneal fluid and ascites.

### SUMMARY

The presently disclosed instrumentalities overcome the problems outlined above and advance the art by providing a nutritional or dietary supplement that improves survivability in a test population of subject who are predisposed to hypertensive heart failure. The diet may be supplemented with a lipid that is an essential fatty acid, especially linoleic acid or a product of linoleic acid, such as cardiolipin (CL), especially CL in unmodified or underivatized form.

In an embodiment, a subject, such as a human, is diagnosed with cardiac disease or a determination is made that the test subject is at risk of cardiac disease. This diagnosis or determination may be for the cardiac disease of hypertensive heart failure. Diagnoses and determinations such as these are within the level of ordinary skill for physicians who are trained in cardiac care. A lipid may be administered to treat actual cardiac disease or as a prophylactic aid against the disease.

Progress or status of treatment may be monitored by assaying a biological indicator in a sample taken from the test subject. The biological indicator may be implicated in CL synthesis, or the processing of linoleic acid, for example, as are the various isoforms of peroxisome proliferator-activated receptors (PPARs). Another useful indicator is the level of cellular ATP. Biological indicators optionally are one or more parameters related to heart disease. Examples of morphological indicators include but are not limited to ventricular wall thickness, cardiac output, left ventricular fractional shortening.

Other related modalities include the administration of linoleic acid to upregulate one or more PPARs, the upregulation of one or more PPARs to increase CL levels, and upregulation of one or more PPARs to reduce production of arachidonic acid. Other modalities include but are not limited to vascular reactivity, inflammatory mediators, cardiolipin, arachidonic acid, antioxidants, delta 5 and delta 6 desaturases, TAZ gene upregulation.

In other aspects, the administration of CL and/or linoleic acid is shown to reduce inflammation, especially cardiac inflammation. These substances may be delivered in a linoleic acid composition including a mixture of lipids, such as a mixture of CL and linoleic acid and/or α-linoleic acid with other fatty acids of Table 1, or as a pure substance.

### BRIEF DESCRIPTION OF THE DRAWINGS

FIG. 1 shows a general structure of singly ionized CL.

FIG. 2 diagrams a pathway of CL synthesis in vivo.

FIG. 3 shows in vivo remodeling of CL on two alternate pathways to form (18:2)₄CL.

FIG. 4 shows the cardiolipin profile of interfibrillar mitochondria (FIGS. 4A and 4C), and subsarcolemmal mitochondria (FIGS. 4B, 4D, and 4F) taken from SHHF rats of various ages.

FIG. 5 compares cardiolipin content in human male patients who have failing (HF) and nonfailing (NF) hearts including the cardiolipin species (18:2)₄CL (FIG. 5A) and (18:2)₃(20:4)CL (FIG. 5B).

FIG. 6 provides a Pearson correlation between (18:2)₄CL and relative heart weight (FIG. 6A) or serum natriuretic peptide (ANP; FIG. 6B) in TAB failing and sham/non-failing hearts.

FIG. 7 shows comparative survival curves for SHHF rats placed on high or low linoleic acid diets.

FIG. 8 provides comparative results for CL content in human failing and non-failing hearts (FIG. 8A) in context of SHHF rats of various ages (FIG. 8B).

FIG. 9 shows an analysis of CL from SHHF heart tissue samples.

FIG. 10 shows percent change of SHHF left ventricles (LVs) mRNA for CL synthase (CLS) and taffazin (TAZ) genes.

FIG. 11 shows Δ6 desaturase mRNA levels in female SHHF LVs from two month old and heart failure rats.

FIG. 12 demonstrates alterations in fractional shorterning and CL in the presence of the Δ5 desaturase and Δ6 desaturase inhibitor, SC-26196 (SC).

FIG. 13 demonstrates PPARα and PPARβ binding in LVs of twenty month old SHHF rats on diets comprising a linoleic acid supplement.

FIG. 14 shows creatine kinase activity in young and failing SHHF rat heart mitochondria.

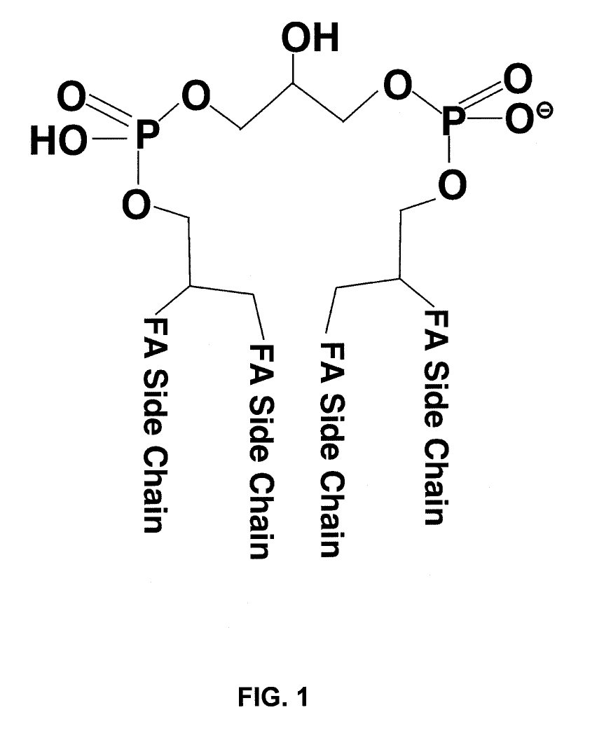

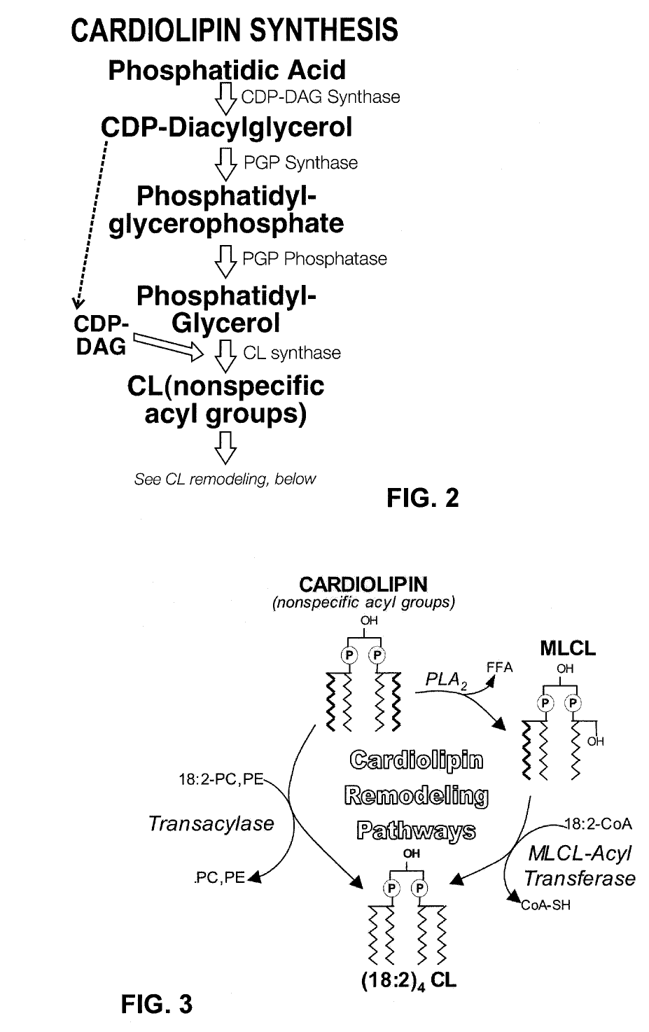

### DETAILED DESCRIPTION

An “effective amount” is intended to qualify the amount of active ingredient that will achieve the goal of fewer or less intense symptoms associated with a cardiac disease. “Effective” may also refer to improvement in disorder severity or the frequency of incidence over no treatment.

For purposes of this disclosure, a “subject” may be selected from rodents, bovine, ovine, avian, equine, porcine, caprine, leporine, feline, canine, humans and primates. In a preferred embodiment, a subject is a human.

A “fatty acid” is a carboxylic acid that generally has a long unbranched aliphatic carbon chain. Cardiolipin (CL) has a double glycerophosphate backbone and two fatty acid side chains. The designations (18:2), (18:1), (18:3), (16:1), (20:4) etc., refer to the number of carbon atoms in the fatty acid chain and the number of double bonds therein, respectively. For example, linoleic acid (18:2) contains 18 carbon atoms and 2 double bonds. Exemplary fatty acids include:

omega-3 fatty acids such as:
alpha-linolenic acid (CH₃(CH₂CH═CH)₃(CH₂)₇COOH)

omega-6 fatty acids such as:
linoleic acid (CH₃(CH₂)₄CH═CHCH₂CH═CH(CH₂)₇COOH)
arachidonic acid (CH₃(CH₂)₄(CH═CHCH₂)₄(CH₂)₂COOH)

omega-9 fatty acids such as:
oleic acid (CH₃(CH₂)₇CH═CH(CH₂)₇COOH)

and/or saturated fatty acids such as:
palmitic acid (CH₃(CH₂)₁₄COOH)
stearic acid (CH₃(CH₂)₈COOH).

Generally, linoleic acid is available as an oil, which may be used directly, e.g., as drops or in gelatin capsules; mixed with binders and pressed into pills; added to water or other consumable fluids, and optionally an emulsifying agent, to form a suspension; mixed with thickeners to form syrups; mixed with rubber to form chewing gum; mixed with liquefied sugars to form lozenges; aerosolized to form sprays; etc. As such, the disclosed compositions may be administered orally, e.g., as a pill, powder, suspension, syrup, lozenge, spray or gum, or nasally, e.g., as an aerosol spray or mist.

Cardiolipin is clearly important for normal mitochondrial function. Mitochondrial dysfunction and impaired oxidative energy production is a contributing factor to heart failure. CL content and composition are radically altered in HF. Aberrant CL synthesis and/or remodeling directly contributes to the inability of the failing heart to generate sufficient energy to sustain normal function. Identification of the influence of linoleic acid-rich and linoleic acid-poor diets on CL maintenance in HF facilitates the design of simple, inexpensive dietary interventions that may positively modulate these processes.

The use of CL and/or linoleic acid to treat heart disease or as a prophylactic against heart disease is disclosed herein. This treatment improves survivability in a test population of subjects who are predisposed to hypertensive heart failure. A diet may be supplemented with a lipid that is an essential fatty acid, and especially linoleic acid or a product of linoleic acid, such as cardiolipin (CL), especially CL in unmodified or underivatized form (18:2)₄CL. Linoleic acid may be administered as a percentage of daily caloric intake. For example, linoleic acid may comprise at least three percent of daily caloric intake, or between three and ten percent of daily caloric intake, or between three to five percent of daily caloric intake.

In an embodiment, cardiolipin may be administered in combination with linoleic acid. Cardiolipin, which may be isolated from cow heart mitochondrial membranes or bacterial cell membranes, can be purchased from chemical suppliers, such as Sigma-Aldrich. Commercially available cardiolipin comprises predominantly linoleate sidechains. It may, however, be desirable to administer cardiolipin and linoleic acid in a cardiolipin:linoleic acid ratio of between 1:0.25 to 1:10, or between 1:0.5 to 1:5, or between 1:1 to 1:4, such as 1:1, 1:2, 1:3 or 1:4.

It is also shown that progress or status of treatment may be monitored by assaying a biological indicator in a sample taken from the test subject. The biological indicator may be implicated in CL synthesis, or the processing of linoleic acid, for example, as are the various isoforms of peroxisome proliferator-activated receptors (PPARs). Another useful indicator is the level of cellular ATP. Particularly relevant indicators include those related to heart failure, typically via echocardiographic measurements.

Other related modalities of treatment or prophylaxis include the administration of linoleic acid to upregulate one or more PPARs, the upregulation of one or more PPARs to increase CL levels, and upregulation of one or more PPARs to reduce production of arachidonic acid.

In other aspects, the administration of CL and/or linoleic acid is shown to reduce inflammation, especially cardiac inflammation. These substances may be delivered in a linoleic acid composition including a mixture of lipids, such as a mixture of CL and linoleic acid and/or α-linoleic acid with other fatty acids of Table 1. In an alternate embodiment, linoleic acid may be administered in a pure form, i.e., 100% linoleic acid.

Administration may occur once daily, or be divided into several smaller doses over the course of 24 hours, e.g., half of the recommended dose may be administered twice daily, a third of the recommended dose may be administered every 8 hours, a quarter of the recommended dose may be administered every 6 hours, a sixth of the recommended dose may be administered every 4 hours, a twelfth of the recommended dose may be administered every 2 hours or a twenty-fourth of the recommended dose may be administered every hour. In a preferred embodiment, administration occurs once daily.

The non-limiting examples that follow teach by way of example to illustrate preferred embodiments, and should not be construed in a manner that unduly limits the scope of the compositions and methods disclosed herein.

### EXAMPLES

### Markers and Methods for Monitoring Linoleic Acid Treatment

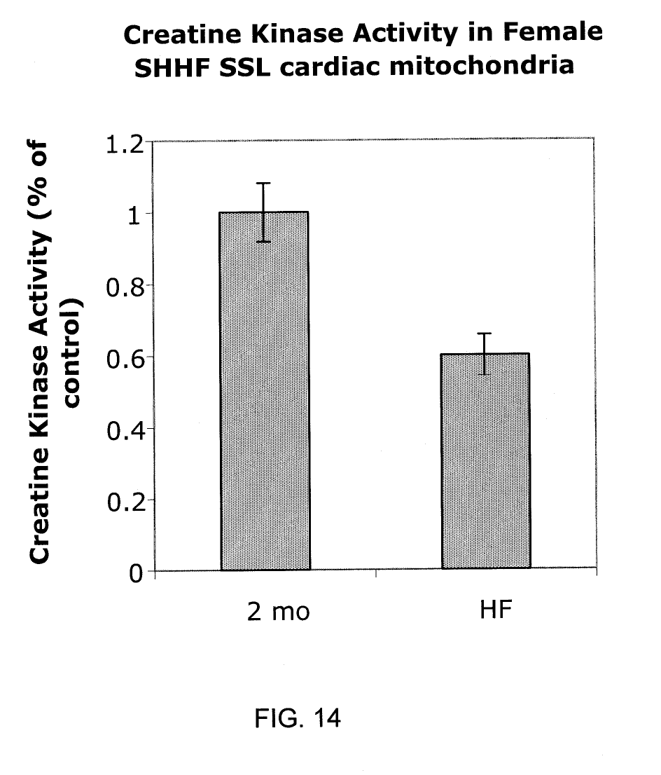

Creatine Kinase: Creatine kinase activity is an indirect measure of the amount of ATP available and mitochondrial creatine kinase activity is dependent on the presence of functional CL. Measurements of creatine kinase activity in young and failing SHHF rat heart mitochondria reveal a significant decrease in CK activity (FIG. 14).

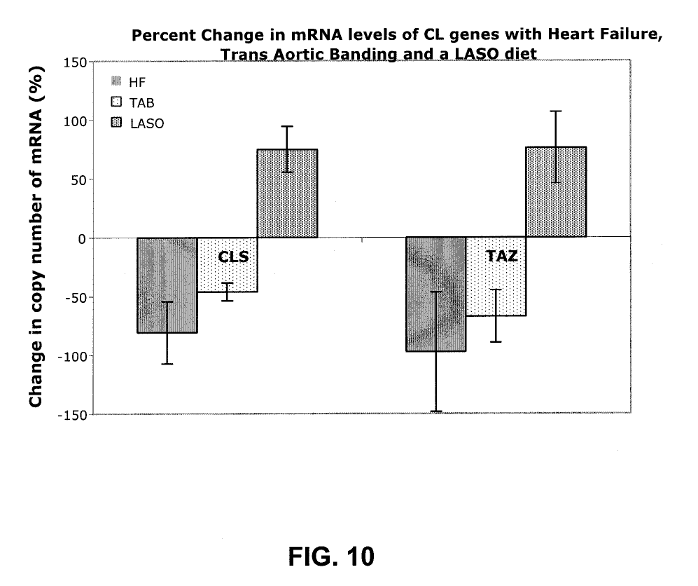

CL Synthase and Tafazzin (TAZ): Genetic markers such as mRNA from genes involved in CL synthesis and remodeling are useful diagnostic tools for assessing linoleic acid supplementation efficacy. FIG. 10 shows percent change in mRNA levels of a synthesis gene (CL Synthase, CLS) and remodeling gene (TAZ) using qrtPCR. This figure shows that both of these genes are reduced in heart failure and also in heart failure induced in young animals using transaortic banding (TAB). A linoleic acid and safflower oil (LASO) diet comprising 10% linoleic acid, results in an increase of both TAZ and CLS compared to control rats of the same age.

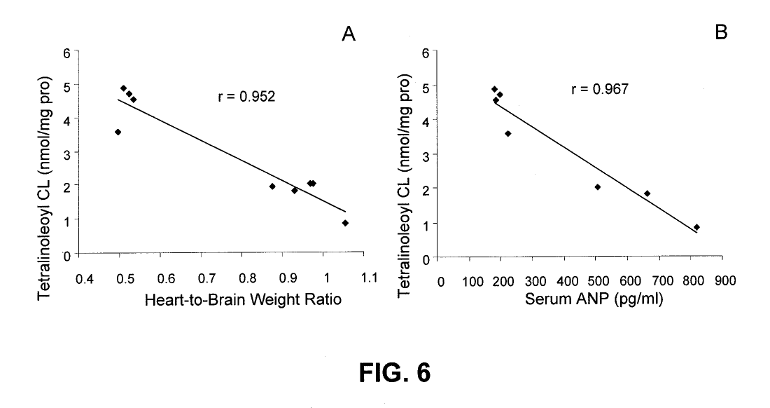

Atrial Natriuretic Peptide: Atrial natriuretic peptide (ANP) is a polypeptide hormone secreted by atrial myocytes that is involved in the homeostatic control of body water, sodium, potassium and adiposity. It is released by atrial myocytes, muscle cells in the atria of the heart, in response to high blood pressure. ANP acts to reduce the water, sodium and adipose loads on the circulatory system, thereby reducing blood pressure. FIG. 6B shows a significant correlation between (18:2)₄CL and serum ANP levels in rapid induction of HF rats. This well established marker of HF strongly correlates with the levels of functional (18:2)₄CL.

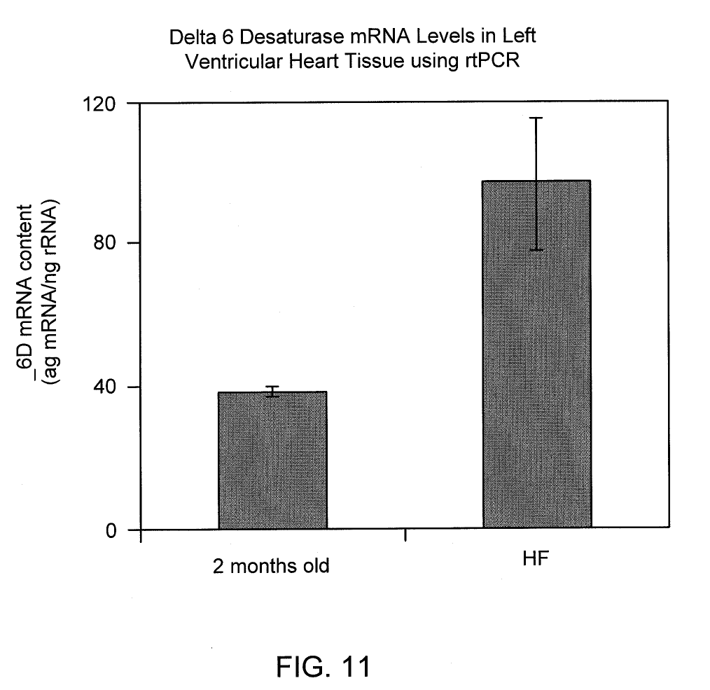

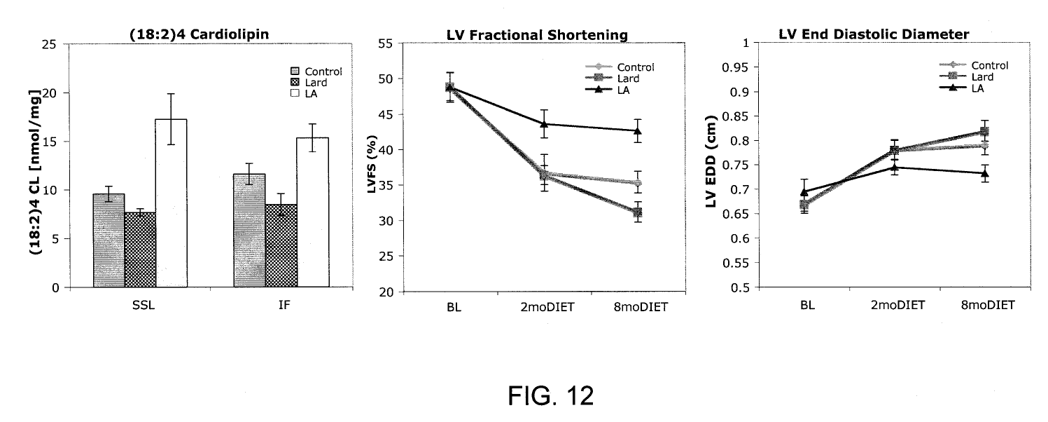

Δ5 Desaturase and Δ6 Desaturase: Δ6 desaturase mRNA increases with heart failure in SHHF rats (FIG. 11). The Δ5 desaturase and Δ6 desaturase inhibitor SC-26196 (Pfizer) was fed to TAB rats in which heart failure was induced three weeks after TAB. The rats were allowed to reach a pre-failure state for a week and then the drug, SC-26196, was given in the chow for two weeks. SC-26196, effectively blocked the production of arachidonic acid from linoleic acid, improved cardiac function (FIG. 12A), increased the level of (18:2)₄CL (FIG. 12B) and improved survivability of SHHF rats. Use of the Δ5 desaturase and Δ6 desaturase inhibitor, SC-26196, improved the heart function of SHHF rats and caused an increase in linoleic acid and a decrease in arachidonic acid levels in the heart.

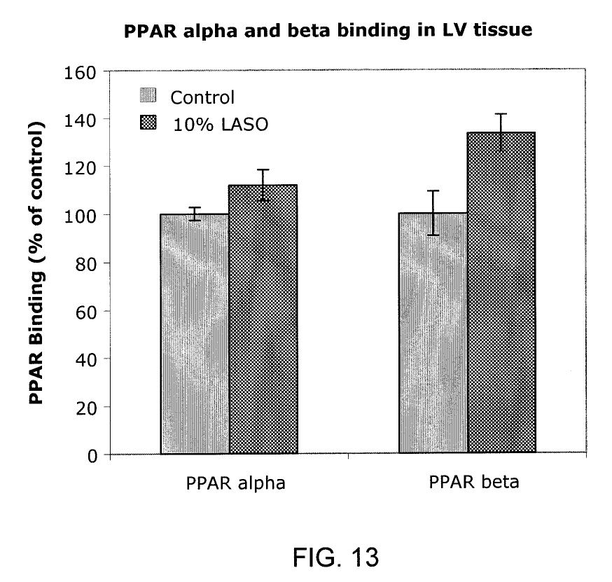

Peroxisome Proliferator-Activated Receptors: Peroxisome proliferator-activated receptors (PPARs) are a family of nuclear transcription factors comprised of PPARα, PPARγ, and PPARβ (also called PPARδ). They have been shown to modulate genes that regulate lipid and glucose metabolism. Alteration in PPAR isoform levels results in CL changes, and it is well established that the natural activator for PPARs are polyunsaturated fatty acids, such as 18:2. Furthermore, PPARs are downregulated during pathological hypertrophy leading to HF. PPARβ is currently believed to be the most pharmacologically promising of the three isoforms. Linoleic acid supplementation results in an increase in PPARβ binding, FIG. 13.

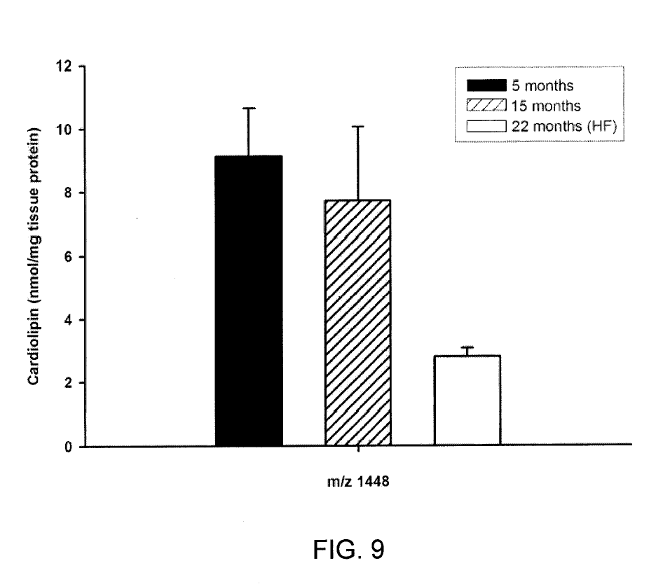

Fractional Shortening and End Diastolic Diameter. Fractional shortening and end diastolic diameter are both common means of assessing heart health. The effects of long term supplementation with linoleic acid safflower oil (LASO), results in stabilization of (18:2)₄CL, LV fractional shortening, and end diastolic diameter. In contrast, rats fed both the control and lard diets experienced a decrease in heart function over time (FIGS. 9B and 9C).

### Experimentation:

Experiments were performed to determine whether the amount of (18:2)₄CL in vivo may be altered with diet and, if so, whether this capability may be used to influence the time course of heart failure in SHHF rats. The two diets studied were a linoleic acid-rich safflower oil diet and a diet supplemented with lard. A highly saturated fat (“linoleic acid-poor” diet) has been shown to be a substandard substrate for CL synthase in neonatal cardiomyocytes [4]. The results show that a linoleic acid-rich diet may delay the onset of HF and improve cardiac mitochondrial function due to positive alterations in the composition of cardiac cardiolipin.

Lean male SHHF rats aged eight weeks and fifteen months were used in the studies. The lean male SHHF rat model was chosen because it demonstrates a clearly reproducible heart failure state (at eighteen to twenty-two months), which is well characterized. The progression toward heart failure in SHHF rats shares a wide variety of marked similarities to the progression toward heart failure in humans.

To study the normal timecourse of HF, male SHHF rats were placed on special diets for six months, from fifteen to twenty-one months of age. This timecourse was chosen, because fifteen months is the time when SHHF rats start to progress from hypertension to HF. By twenty-one months most controls should be showing signs of overt HF.

Rapid induction of HF was studied using eight week old male SHHF rats maintained on a control (Purina 5001) diet since weaning from their mothers. Rapid HF is induced by transaortic banding (TAB) accompanied by a high salt diet for four weeks (TAB/HS) [47]. Rats are used for experiments four weeks following TAB/HS treatment.

These experiments, (i) characterize in detail the changes in CL content and composition that occur in response to dietary changes during the transition to uncompensated HF, (ii) determine the extent to which CL changes influence HF outcomes, and (iii) investigate the role of PPARs and taffazin (TAZ) genes in the SHHF rat model.

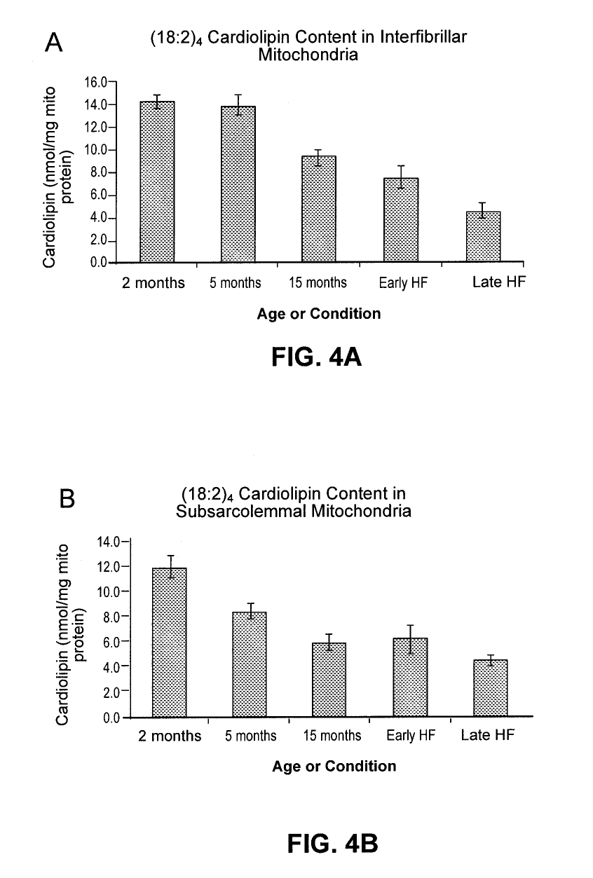

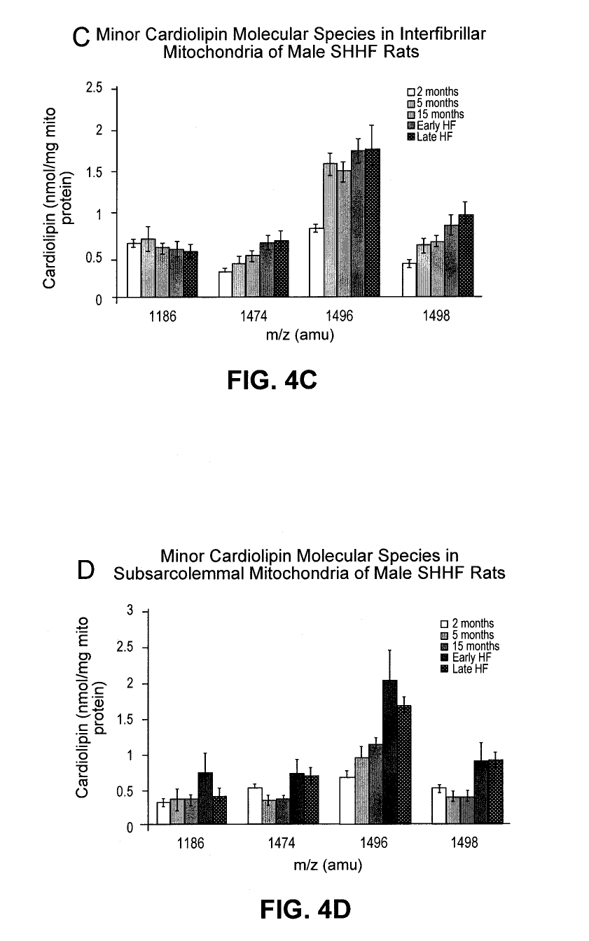

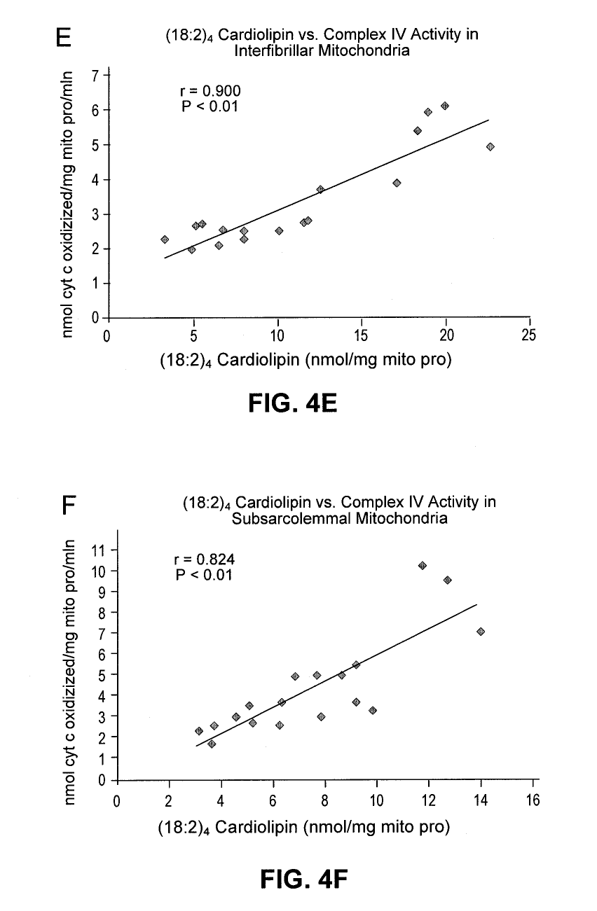

In order to provide a thorough examination of any CL alterations that may occur in the failing heart, CL molecular subspecies profiles in cardiac subsarcolemmal and interfibrillar mitochondria isolated from male SHHF rats were studied using mass spectrometry [3]. Male SHHF rats develop hypertension early in life (at three to four months of age), begin exhibiting signs of HF by about fifteen months of age, and typically progress to overt HF by twenty-two to twenty-three months of age. In this study, a marked reduction of (18:2)₄CL in both types of mitochondria from failing hearts was observed. This marked reduction was accompanied by substantial increases in non-(18:2)₄CL subspecies [3]. These experiments demonstrate a strong positive correlation between the (18:2)₄CL levels and cytochrome oxidase activity, as shown in FIG. 4. These data provide further support of the role of (18:2)₄CL in mitochondrial respiratory function.

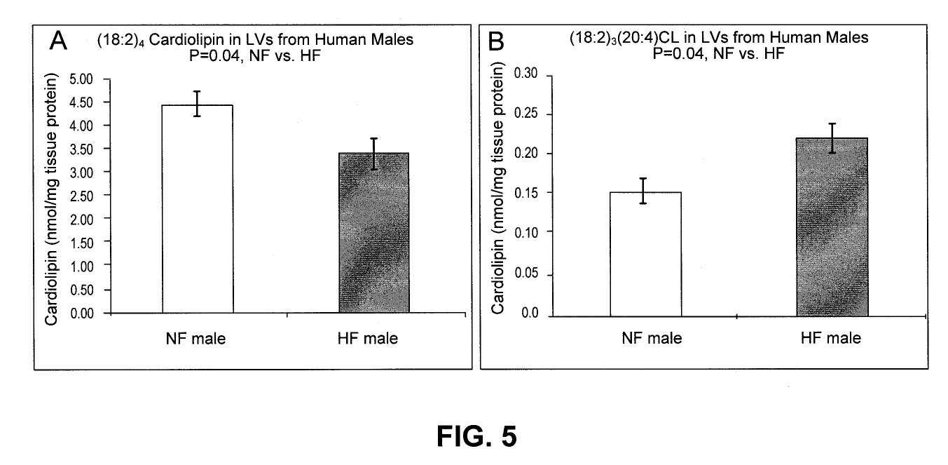

In addition to mitochondria isolated from male SHHF rats, mitochondria from human LV tissue was tested for CL content. FIG. 5 shows that in human LV tissue, a marked reduction of (18:2)₄CL in both types of mitochondria from failing hearts was observed. (18:2)₄CL decreases in HF while alternative molecular species of CL increase.

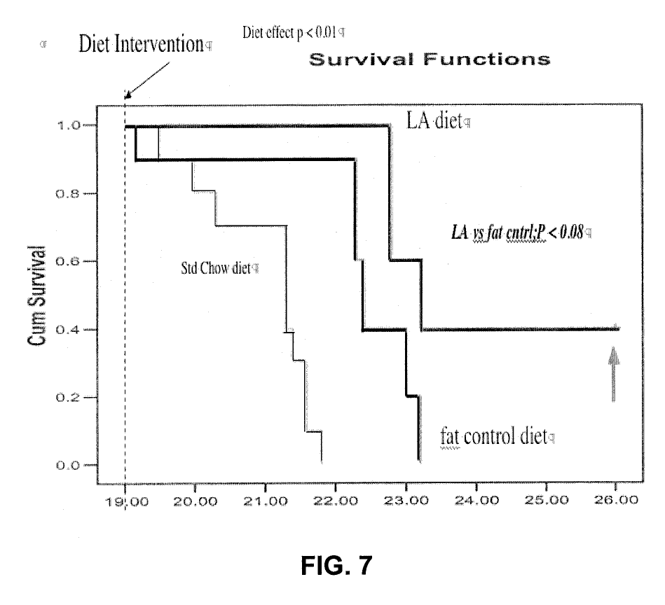

FIG. 7 demonstrates that the diet rich in linoleic acid substantially improves survivability of SHHF rats, as compared to the diet supplemented with lard. Improved survival occurs in the absence of a reduction in blood pressure or differences in caloric intake. Rats exhibit extended lifespans greater than twenty-six months compared to rats receiving lard supplemented and standard diets. This extended survival is unprecedented.

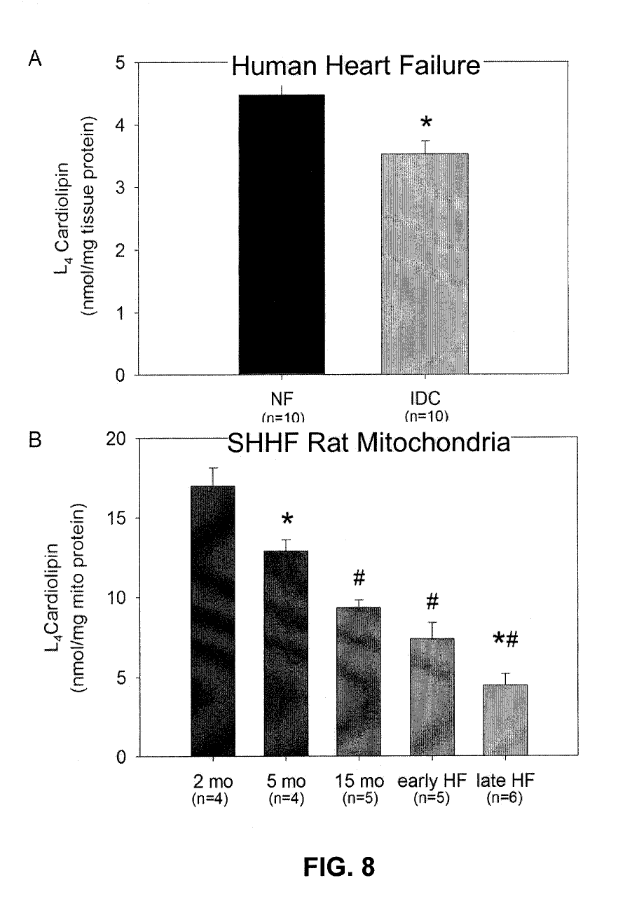

FIG. 8 shows that (18:2)₄ cardiolipin decreases in human heart tissue during idiopathic cardiomyopathy (IDC) compared to nonfailing (NF) controls (FIG. 8A), and SHHF rat heart interfibrillar mitochondria (FIG. 8B).

The results show that cardiolipin is clearly important for normal mitochondrial function. Mitochondrial dysfunction and impaired oxidative energy production is a contributing factor to heart failure. CL content and composition are radically altered in HF, aberrant CL synthesis and/or remodeling may directly contribute to the inability of the failing heart to generate sufficient energy to sustain normal function. Identification of the influence of linoleic acid-rich and linoleic acid-poor diets on CL maintenance in HF facilitates the design of simple, inexpensive dietary interventions that may positively modulate these processes. These results may provide treatments for and diagnosis of heart disease.

These data reveal a strong correlation between alterations in the 18:2 content of the mitochondrial phospholipid, cardiolipin (CL), in the heart and the development of HF. 18:2 supplementation results in a significant increase in survival with the 18:2 diet over either the lard or standard chow diets. Trends for improvement of left ventricular dimension and systolic function are also seen.

Changes may be made in the above compositions and methods without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present methods and compositions, which, as a matter of language, might be said to fall there between.

### REFERENCES

The following references are hereby incorporated by reference to the same extent as though fully replicated herein:
1. Katz, A. M., Is the failing heart energy depleted? Cardiol. Clin., 1998. 16: p. 633-644.
2. Ingwall, J. S. and R. G. Weiss, Is the failing heart energy starved? On using chemical energy to support cardiac function. Circ Res, 2004. 95(2): p. 135-45.
3. Sparagna, G. C., et al., Quantitation of cardiolipin molecular species in spontaneously hypertensive heart failure rats using electrospray ionization mass spectrometry. J Lipid Res, 2005. 46(6): p. 1196-204.
4. Ostrander, D. B., et al., Decreased cardiolipin synthesis corresponds with cytochrome c release in palmitate-induced cardiomyocyte apoptosis. J. Biol. Chem, 2001. 276: p. 38061-38067.
5. Hatch, G. M., Cardiolipin: Biosynthesis, remodeling, and trafficking in the heart and mammalian cells. Intl. J. Mol. Med., 1998. 1: p. 33-41.
6. Hoch, F. L., Cardiolipins and biomembrane function. Biochim. Biophys. Acta, 1992. 1113: p. 71-133.
7. Zhang, C. L., V. Lyngmo, and A. Nordoy, The effects of saturated fatty acids on endothelial cells. Thromb. Res., 1992. 65: p. 65-75.
8. Lange, C., et al., Specific roles of protein-phospholipid interactions in the yeast cytochrome be1 complex structure. Embo J, 2001. 20(23): p. 6591-600.
9. Tuominen, E. K. J., C. J. A. Wallace, and P. K. J. Kinnunen, Phospholipid-cytochrome c interaction. Evidence for the extended lipid anchorage. J. Biol. Chem, 2002. 277: p. 8822-8826.
10. McMillin, J. B. and W. Dowhan, Cardiolipin and apoptosis. Biochim. Biophys. Acta, 2002. 1585(2-3): p. 97-107.
11. Schlame, M., L. Horvath, and L. Vigh, Relationship between lipid saturation and lipid-protein interaction in liver mitochondria modified by catalytic hydrogenation with reference to cardiolipin molecular species. Biochem J, 1990. 265(1): p. 79-85.
12. Yamaoka, S., R. Urade, and M. Kito, Mitochondrial function in rats is affected by modification of membrane phospholipids with dietary sardine oil. J Nutr, 1988. 118(3): p. 290-6.

13. Hostetler, K. Y., et al., Further studies on the formation of cardiolipin and phosphatidylglycerol in rat liver mitochondria. Effect of divalent cations and the fatty acid composition of CDP-diglyceride. Biochim Biophys Acta, 1975. 380(3): p. 382-9.
14. Rustow, B., et al., Species pattern of phosphatidic acid, diacylglycerol, CDP-diacylglycerol and phosphatidylglycerol synthesized de novo in rat liver mitochondria. Biochim Biophys Acta, 1989. 1002(2): p. 261-3.
15. Schlame, M. and B. Rustow, Lysocardiolipin formation and reacylation in isolated rat liver mitochondria. Biochem J, 1990. 272(3): p. 589-95.
16. Xu, Y., et al., Remodeling of cardiolipin by phospholipid transacylation. J. Biol. Chem, 2003. 278: p. 51380-51385.
17. Vreken, p., et al., Defective remodeling of cardiolipin and phosphatidylglycerol in Barth syndrome. Biochem. Biophys. Res. Comm., 2000. 279: p. 378-382.
18. Schlame, M., et al., Deficiency of tetralinoleoyl-cardiolipin in Barth syndrome. Ann Neurol, 2002. 51(5): p. 634-7.
19. Valianpour, F., et al., Linoleic acid supplementation of Barth syndrome fibroblasts restores cardiolipin levels: implications for treatment. J Lipid Res, 2003. 44(3): p. 560-6.
20. Lesnefsky, E. J., et al., Myocardial ischemia selectively depletes cardiolipin in rabbit heart subsarcolemmal mitochondria. Am. J. Physiol. Heart Circ. Physiol., 2001. 280: p. H2770-H2778.
21. O'Rourke, B. and D. K. Reibel, Effects of adrenoceptor blockade on cardiac hypertrophy and myocardial phospholipids. Proc Soc Exp Biol Med, 1992. 200(1): p. 95-100.
22. Nasa, Y., et al., Changes in fatty acid compositions of myocardial lipids in rats with heart failure following myocardial infarction. Mol Cell Biochem, 1997. 176(1-2): p. 179-89.
23. Heerdt, P. M., et al., Disease-specific remodeling of cardiac mitochondria after a left ventricular assist device. Ann. Thorac. Surg., 2002. 73: p. 1216-1221.
24. Knapp, H. R., Dietary fatty acids in human thrombosis and hemostasis. Am J Clin Nutr, 1997. 65(5 Suppl): p. 1687S-1698S.
25. Storlien, L. H., et al., Does dietary fat influence insulin action? Ann N Y Acad Sci, 1997. 827: p. 287-301.
26. Calder, P. C., Polyunsaturated fatty acids, inflammation, and immunity. Lipids, 2001. 36(9): p. 1007-24.
27. Lahoz, C., et al., Effects of dietary fat saturation on eicosanoid production, platelet aggregation and blood pressure. Eur J Clin Invest, 1997. 27(9): p. 780-7.
28. Charnock, J. S., et al., Dietary fats and oils in cardiac arrhythmia in rats. Am J Clin Nutr, 1991. 53(4 Suppl): p. 1047S-1049S.
29. Laaksonen, D. E., et al., Serum fatty acid composition predicts development of impaired fasting glycaemia and diabetes in middle-aged men. Diabet Med, 2002. 19(6): p. 456-64.
30. Laaksonen, D. E., et al., Prediction of cardiovascular mortality in middle-aged men by dietary and serum linoleic and polyunsaturated fatty acids. Arch Intern Med, 2005. 165(2): p. 193-9.
31. Huss, J. M. and D. P. Kelly, Nuclear receptor signaling and cardiac energetics. Circ Res, 2004. 95(6): p. 568-78.
32. Jiang, Y. J., et al., Stimulation of cardiac cardiolipin biosynthesis by PPARalpha activation. J Lipid Res, 2004. 45(2): p. 244-52.
33. Watkins, S. M., et al., Lipid metabolome-wide effects of the PPARgamma agonist rosiglitazone. J Lipid Res, 2002. 43(11): p. 1809-17.
34. McCune, S. A., et al., The SHHF/Mcc-fa^{cp}: A genetic model of congestive heart failure, in Mechanisms of Heart Failure, P. K. Singal, et al., Editors. 1995, Kluwer Academic Publishers: Boston. p. 91-106.
35. Ishizuka, T., et al., Phonotypic consequences of a nonsense mutation in the leptin receptor gend (fa^{K}) in obese spontaneously hypertensive Koletsly rats (SHROB). J. Nutr., 1998. 128: p. 2299-2306.
36. Bergman, M. R., et al., Myocardial tumor necrosis factor-alpha secretion in hypertensive and heart failure-prone rats. Am J Physiol, 1999. 277(2 Pt 2): p. H543-50.
37. Carraway, J. W., et al., Comparison of irbesartan with captopril effects on cardiac hypertrophy and gene expression in heart failure-prone male SHHF/Mcc-fa(cp) rats. J Cardiovasc Pharmacol, 1999. 33(3): p. 451-60.
38. Kogler, H., et al., Disproportionate enhancement of myocardial contractility by the xanthine oxidase inhibitor oxypurinol in failing rat myocardium. Cardiovasc Res, 2003. 59(3): p. 582-92.
39. Onodera, T., et al., Maladaptive remodeling of cardiac myocyte shape begins long before failure in hypertension. Hypertension, 1998. 32(4): p. 753-7.
40. Park, S. C., et al., Comparison of verapamil and felodipine treatment on lipid and glucose metabolism in obese female SHHF/Mcc-facp rats. Proc Soc Exp Biol Med, 1999. 221(3): p. 224-33.
41. Pocchiari, R. J., R. L. Hamlin, and S. A. McCune, Electrocardiographic findings in rats with cardiomyopathy. Am J Vet Res, 1993. 54(4): p. 607-11.
42. Radin, M. J., et al., Effects of enalapril and clonidine on glomerular structure, function, and atrial natriuretic peptide receptors in SHHF/Mcc-cp rats. J Cardiovasc Pharmacol, 1992. 19(3): p. 464-72.
43. Radin, M. J., et al., Increased salt sensitivity secondary to leptin resistance in SHHF rats is mediated by endothelin. Mol Cell Biochem, 2003. 242(1-2): p. 57-63.
44. Sack, M. N., et al., Mitochondrial fatty acid oxidation enzyme gene expression is downregulated in the failing heart. Circulation, 1996. 94: p. 2837-2842.
45. Tamura, T., et al., Temporal regression of myocyte hypertrophy in hypertensive, heart failure-prone rats treated with an AT1-receptor antagonist. J Card Fail, 2002. 8(1): p. 43-7.
46. Paradies, G., et al., Age-dependent decline in the cytochrome c oxidase activity in rat heart mitochondria. role of cardiolipin. FEBS Lett., 1997. 406: p. 136-138.
47. McCune, S. A., et al., Rapid onset of heart failure in male SHHF rats induced by high salt diet and thoracic aortic banding. J Mol Cell Cardio, 2004. 36: p. 621.
48. Fannin, S. W., et al., Aging selectively decreases oxidative capacity in rat heart interfibrillar mitochondria. Arch. Biochem. Biophys., 1999. 372: p. 399-407.
49. Palmer, J. W., B. Tandler, and C. L. Hoppel, Biochemical properties of subsarcolemmal and interfibrillar mitochondria isolated from rat cardiac muscle. J. Biol. Chem, 1977. 252: p. 8731-8739.
50. Hickson-Bick, D. L., et al., Palmitate-induced apoptosis in neonatal cardiomyocytes is not dependent on the generation of ROS. Am. J. Physiol. Heart Physiol., 2002. 282: p. H656-H664.
51. Iemitsu, M., et al., Aging-induced decrease in the PPAR-alpha level in hearts is improved by exercise training. Am J Physiol Heart Circ Physiol, 2002. 283(5): p. H1750-60.
52. Garnier, A., et al., Depressed mitochondrial transcription factors and oxidative capacity in rat failing cardiac and skeletal muscles. J Physiol, 2003. 551(Pt 2): p. 491-501.
53. Moore, R. L., et al., Altered Ca2+ dynamics in single cardiac myocytes from renovascular hypertensive rats. Am J Physiol, 1991. 260(2 Pt 1): p. C327-37.
54. Emter, C. A., et al., Low-Intensity Exercise Training Delays the Onset of Decompensated Heart Failure in the Spontaneously Hypertensive Heart Failure (SHHF) Rat. Am J Physiol, 2005. Submitted for Publication.
55. Holycross, B. J., et al., Plasma renin activity in heart failure prone (SHHF/Mcc-fa^{cp}) rats. Am. J. Physiol., 1997. 42: p. H228-H233.
56. Jew, K. N. and R. L. Moore, Glibanclamide improves postischemic recovery of myocardial contractile function in trained and sedentary rats. J. Appl. Physiol., 2001. 91: p. 1545-1554.
57. Warren, C. M. and M. L. Greater, Method for cardiac myosin heavy chain separation by sodium dodecyl sulfate gel electrophoresis. Anal Biochem, 2003. 320(1): p. 149-51.
58. Sparagna, G. C., et al., A metabolic role for mitochondria in palmitate-induced cardiac myocyte apoptosis. Am. J. Physiol. Heart Circ Physiol., 2000. 279: p. H2124-H2132.
59. Ragan, C. I., et al., Sub-fractionation of mitochondria and isolation of the proteins of oxidative phosphorylation, in Mitochondria, A Practical Approach, V. M. Darley-Usmar, D. Rickwood, and M. T. Wilson, Editors. 1987, IRL Press: Oxford. p. 96-101.
60. Gudz, T. I., K.-Y. Tserng, and C. L. Hoppel, Direct inhibition of mitochondrial respiratory chain complex III by cell-permeable ceramide. J. Biol. Chem., 1997. 272: p. 24154-24158.

What is claimed:

1. A method of treating a subject to produce a cardiac benefit, comprising:
diagnosing a subject animal that is selected from the group consisting of animals who are at risk of developing a cardiac disease, and animals who have a cardiac disease; and
administering a composition of linoleic acid to elevate at least one of linoleic acid and tetra-linoleoyl species of cardiolipin ((18:2)₄CL) in the subject.

2. The method of claim 1 wherein the animal is a human.

3. The method of claim 2 wherein the step of diagnosing includes self-diagnosis by the human.

4. The method of claim 1 wherein the step of diagnosing includes observing a symptom of a disease selected from hypertension, ischemic cardiomyopathy, heart disease and metabolic syndrome.

5. The method of claim 1 wherein the step of diagnosing includes diagnosing a subject who is at risk for heart disease.

6. The method of claim 1 wherein the composition further comprises cardiolipin.

7. The method of claim 6 wherein the cardiolipin and the linoleic acid are present in a ratio between 1:0.25 to 1:10.

8. The method of claim 1 further including a step of monitoring a status of treatment by measuring levels of a biological indicator in a sample taken from the subject.

9. The method of claim 8 wherein the biological indicator is implicated in (18:2)₄CL synthesis or the processing of linoleic acid.

10. The method of claim 8 wherein the biological indicator is selected from cardiolipin levels in blood leukocytes and a level of cellular ATP.

11. The method of claim 8 wherein the step of monitoring includes observing an increase in binding of a PPAR in combination with an increase in cardiolipin.

12. The method of claim 8 wherein the step of monitoring includes monitoring a level of at least one of arachidonic acid, inflammation, C reactive protein, atrial natriuretic peptide, brain natriuretic peptide, urine protein, triglycerides and insulin.

13. The method of claim 8 wherein the step of monitoring includes assaying to confirm inhibition of Δ5 desaturase and/or Δ6 desaturase.

14. The method of claim 1 further comprising a step of monitoring status of treatment by assaying a morphological indicator of the subject.

15. The method of claim 14 wherein the morphological indicator comprises left ventricular wall thickness.

16. The method of claim 1 further comprising a step of monitoring status of treatment by measuring cardiac output.

17. The method of claim 16 wherein the cardiac output is selected from ventricular stroke volume and fractional shortening.

18. A composition for use against cardiac disease, comprising:
an effective amount of linoleic acid for treating cardiac disease as measured by a comparatively improved survivability in a population of test subjects.

19. The composition of claim 18 wherein the effective amount includes a dosage that is effective against active cardiac disease.

20. The composition of claim 18 wherein the effective amount includes a dosage that is effective in prophylaxis against cardiac disease.

21. The composition of claim 18 wherein the effective amount comprises at least three percent of daily caloric intake.

22. The composition of claim 18 wherein the linoleic acid is present at a concentration of 75-85%.

23. The composition of claim 18 further comprising cardiolipin.

24. The composition of claim 23 wherein the cardiolipin and the linoleic acid are present in a ratio between 1:0.25 to 1:10.
